(19) United States
(12) Patent Application Publication
(10) Pub. No.: US 2014/0276442 A1
(43) Pub. Date: Sep. 18, 2014

(54) DUAL CHAMBER DEVICE

(71) Applicant: James Q. Haughey, Newry (GB)

(72) Inventor: James Q. Haughey, Newry (GB)

(73) Assignee: NORBROOK LABORATORIES LIMITED, Newry (GB)

(21) Appl. No.: 14/237,313

(22) PCT Filed: Aug. 6, 2012
(86) PCT No.: PCT/GB2012/051900

§ 371 Date: May 27, 2014

(30) Foreign Application Priority Data
Aug. 5, 2011 (GB) 1113606.6

(51) Int. Cl. A61D 7/00; A61M 5/19

(52) U.S. Cl. CPC A61D 7/00; A61M 5/19; A61M 2250/00
USPC 604/191

(57) ABSTRACT

A device (10) comprises a first barrel defining a first chamber for holding a first composition. The first chamber has at least one side wall and a distal end wall defining a dispensing nozzle (22). At least one flow channel (32) is formed in the at least one side wall and distal end wall, extending across the distal end wall in helical fashion. The device (10) further comprises a second barrel (14) defining a second chamber for holding a second composition. The second chamber has at least one side wall and distal end wall. An annular seal (86) is formed at the distal end of the second barrel (14). The at least one side wall defines at least one orifice (58) proximal to the seal (86). A plunger (16) is operable to drive the second barrel (14) into the first chamber to displace the first composition out through the nozzle (22) and to bring the annular seal (86) into contact with the distal end wall of the first chamber. Thereby, the seal (86) is deformed and allows fluid communication between the orifices (58) and the flow channels (32). The plunger (16) is subsequently movable into the second chamber to displace the second composition out through the orifice (58) and flow channels (32) to the nozzle (22).

The present invention is directed to a dual chamber device for sequentially dispensing two compositions, without the compositions mixing with each other.

In some situations it may be necessary or desirable to dispense two compositions which are not mixed with one another but are delivered directly one after the other. One example is in veterinary medicine for treating infection in the udder of animals such as cattle. It is desirable to first inject an antibiotic composition to treat the infection and then to deliver a teat seal to seal the teat canal to prevent the entry of further bacteria.

Various devices and syringes with two or more chambers are known. Many are used where two compositions must be mixed together before they are delivered. Some designs employ separate barrels either adjacent to one another, or a first cylindrical barrel surrounded by an annular second barrel, with each barrel having a separate plunger. Examples can be found in WO 2007/006030 and US 2004/092864. Other designs include a single barrel and plunger which divides the barrel into two chambers and a bypass passage or valve arrangement so that in certain positions of the plunger the two chambers are connected and the compositions can mix. Typical examples are shown in EP 1759728, US 2003/040701 and WO 99/17820.

Some syringes for sequential delivery of separate compositions are also known. In US 2006/224105 a complex, multi-part arrangement of syringe and valve assembly is provided. In U.S. Pat. No. 6,723,074 a single barrel with a flexible membrane defines a chamber for a first composition. A flexible bag contains a second composition. A plunger forces the bag against the membrane to deliver the first composition. Subsequently a piercing member breaks the bag to deliver a second composition. In U.S. Pat. No. 5,102,388 a single barrel is divided into chambers by stoppers with piercing members. As a plunger is depressed fluid from a first chamber is delivered and a first stopper then pierces the adjacent stopper to allow fluid from the next chamber to be delivered and so on. These are all multi-part devices requiring a combination of materials, detailed parts, and piercing members, adding complexity and cost.

BE 1018797 describes a syringe with an outer barrel providing a first chamber and a piston providing a second chamber. A plunger drives the piston into the first chamber to dispense a liquid from the first chamber. The plunger can then be driven into the second chamber to dispense liquid from the second chamber.

The present invention provides a device comprising:
a first barrel defining a first chamber for holding a first composition, the first chamber having at least one side wall and a distal end wall defining a dispensing nozzle, wherein at least one flow channel is formed in the at least one side wall and distal end wall of the first chamber, extending across the distal end wall from the at least one side wall to the nozzle in helical fashion;
a second barrel defining a second chamber for holding a second composition, the second chamber having at least one side wall and a distal end wall, an annular seal at the distal end of the second barrel, and the at least one side wall of the second chamber defining at least one orifice proximal to the seal; and
a plunger operable to drive the second barrel into the first chamber to displace the first composition out of the first chamber through the nozzle, and to bring the annular seal of the second barrel into contact with the distal end wall of the first chamber, thereby to deform the seal and permit fluid communication between each orifice in the second barrel and each flow channel, wherein the plunger is subsequently moveable into the second chamber to displace the second composition out of the second chamber through each orifice and flow channel to the nozzle.

In this way, a single device can be provided which holds two compositions in separate chambers. The user need only operate a single plunger in order to sequentially dispense the first and second compositions, without the two compositions mixing with one another.

Preferably, a plurality of flow channels are provided, arranged in three equi-spaced groups of flow channels.

Preferably, the at least one side wall of the first and second chambers is a cylindrical wall.

This arrangement aids in efficiently delivering the second composition from the second chamber to the nozzle.

Preferably, the syringe further comprises releasable engagement means to engage the plunger with the second barrel, such that when engaged the plunger and second barrel move as one unit to drive the second barrel into the first chamber, and when disengaged the plunger can move into the second chamber.

In this way, a single stroke of the plunger is operable to deliver the first composition, followed by the second composition.

Preferably, the engagement means is releasable by exerting increased force on the plunger. The engagement means may comprise cooperating structures on an inner surface of the second barrel and an outer surface of the plunger.

Thus, a simple mechanism is provided which is cost effective to manufacture and simple to operate without the user needing to operate separate release means.

Preferably, the syringe also includes a closure member to prevent leakage of the second composition from the second chamber through each orifice until the second barrel is located in the first chamber.

In this way, the second chamber can be charged with the second composition before it is engaged with the first barrel.

Preferably, the closure member comprises a tube sealingly located on the second barrel and the device further comprises retention structures to retain the tube and allow the second barrel to move through the tube into the first chamber when driven by the plunger.

Thus, action of the plunger also automatically moves the second barrel relative to the closure member so that each orifice is no longer closed.

Preferably, the device further comprises a releasable stop means for preventing movement of the plunger into the second chamber until the stop is released. This ensures there cannot be unintentional delivery of the second composition.

In a second aspect, the present invention also provides a device comprising:
a first barrel defining a first chamber for holding a first composition and a nozzle communicating with the first chamber;
a second barrel defining a second chamber for holding a second composition and at least one orifice communicating with the second chamber;
a plunger operable to drive the second barrel into the first chamber thereby to displace the first composition out of the first chamber through the nozzle and to bring each orifice into fluid communication with the nozzle, wherein the plunger is subsequently moveable into the second chamber to displace the second composition out of the second chamber through each orifice and the nozzle; and
further comprising a releasable stop means for preventing movement of the plunger into the second chamber until the stop is released.

In a third aspect, the present invention provides a device comprising:
a first barrel defining a first chamber for holding a first composition and a nozzle communicating with the first chamber;
a second barrel defining a second chamber for holding a second composition and at least one orifice communicating with the second chamber, a plunger operable to drive the second barrel into the first chamber thereby to displace the first composition out of the first chamber through the nozzle and to bring each orifice into fluid communication with the nozzle, wherein the plunger is subsequently moveable into the second chamber to displace the second composition out of the second chamber through each orifice and the nozzle; and
further comprising a tube sealingly locatable on the second barrel so as to cover each orifice, and retention structures operable to fix the tube relative to the first barrel and allow the second barrel to pass through the tube and into the first chamber when driven by the plunger.

The present invention will now be described in detail, by way of example only, with reference to the accompanying drawings in which:

FIG. 1 is a cross section of a device in accordance with one embodiment of the present invention, in its starting position;

FIG. 2 shows the device of FIG. 1 in an intermediate position after delivery of a first composition;

FIG. 3 shows the device of FIG. 1 in a final position after removal of a releasable stop means and delivery of a second composition;

FIG. 4 is a cross section of the outer barrel of the device of FIG. 1;

FIG. 5 is an end view of the barrel of FIG. 4;

FIG. 6 is a side view of the inner barrel of the device of FIG. 1;

FIG. 7 is a cross section of the inner barrel of FIG. 6;

FIG. 8 is an enlarged cross section of the protection ring of the device of FIG. 1;

FIG. 9 is a side view of the plunger of the device of FIG. 1;

FIG. 10 is a cross section of the plunger of FIG. 9; and

FIG. 11 is an enlarged perspective view of the releasable stop means of the device of FIG. 1.

As shown in FIG. 1, a device 10 in accordance with one embodiment of the present invention comprises an outer barrel 12, an inner barrel 14, a plunger 16, and a protection ring 18. The device 10 also comprises a releasable stop means 80, stop 82 and cap 84.

As best seen in FIG. 4, the outer barrel 12 comprises a hollow cylindrical body 20, a dispensing nozzle 22 at the distal end of the body 20 and an open proximal end surrounded by a flange 24.

As shown in FIG. 4, the inner bore of the outer barrel 12 comprises an enlarged diameter portion 26 at the proximal end bounded by a shoulder 28, and a smaller diameter portion 30. In use, the enlarged diameter portion 26 receives and retains the protection ring 18 and the smaller diameter portion 30 defines a first chamber for receiving a first composition.

At the distal end of the outer barrel 12, a number of circumferentially spaced grooves 32 are formed in the inner surface of the cylindrical body 20 and the end wall 34, leading to the nozzle 22. In this example, three sets of grooves are provided. As best seen in FIG. 5, the grooves 32 may extend in a converging, arcuate fashion across the end wall 34 towards the nozzle 22. These helical grooves 32 form flow channels which help the compositions to flow towards the nozzle and empty the syringe.

Referring to FIGS. 6 and 7, the inner barrel 14 also comprises a hollow cylindrical body 36. It has a closed distal end 38 with a central projection 40 on the outer surface and a corresponding recess 42 on the inner surface, and an open proximal end surrounded by a flange 44. The inner bore of the cylindrical body 36 includes a proximal portion 46 of slightly larger diameter and a distal portion 48 of slightly smaller diameter, joined by a shoulder 50. The proximal portion 46 may include a flared mouth 52 at the open end to assist with locating the plunger 16. The proximal portion 46 may also include one or more axially extending ridges or grooves 53 to assist with aligning the plunger 16 as discussed further below. The distal end portion 48 may include a further reduced diameter portion 54 adjacent the end wall 38 to receive the distal end of the plunger 16.

As best seen in FIG. 6, the outer surface of the inner barrel 14 includes a waisted portion 56 of reduced outer diameter close to the distal end. A number of circumferentially spaced orifices 58 are provided in this waisted portion 56 (see FIG. 7). In this example, four equi-spaced orifices 58 are provided, but this number may be varied.

A flexible annular flange 86 is formed around the distal end of the inner barrel 14. The flange 86 projects distally, leaving an annular channel 88 between the flange 86 and the outer surface of the distal end 38. The flange 86 is sufficiently thin that it is flexible and deformable as described further below.

The protection ring 18 is shown in FIG. 8 at an enlarged scale. This is initially located around the distal end of the inner barrel 14 as shown in FIG. 1. The protection ring 18 comprises a tubular body 60 with a flange 62 around the proximal end. The inner surface of the body 60 and outer surface of the inner barrel 14 are provided with cooperating structures 64 such as grooves and ribs, which serve to locate the protection ring 18 on the inner barrel 14. As shown in FIG. 6, on the inner barrel 14 the structures 64 are located proximally of the waisted portion 56, and the flexible flange 86 is distal of it. The protection ring 18 covers this area when located on the inner barrel 14. The structures 64 and the flexible flange 86 serve to seal the protection ring 18 against the inner barrel 14 either side of the orifices 58, to prevent leakage of liquid from the inner barrel 14.

The plunger 16 is shown in FIGS. 9 and 10 and comprises a cylindrical body 66. The proximal end includes a flange 68 against which a user exerts pressure in order to operate the device 10. The flange 68 is illustrated relatively small and flat but in practice this may be extended and formed curved or otherwise ergonomically shaped to enhance comfort and ease of operation for a user, in a similar manner to the proximal end flange 24 of the outer barrel 12 which is preferably enlarged and curved to comfortably and securely fit a user's fingers to help in operating the device 10.

At the distal end of the plunger 16, is a reduced diameter nose portion 72 with a central projection 74. A flexible flange 70 is formed on the outer surface of the plunger 16 around the proximal end of the nose portion 72. In a similar manner to the flexible flange 86 on the inner barrel 14, the flange 70 projects distally, leaving an annular channel between it and the nose portion. As seen in FIGS. 1-3, when the plunger 16 is located in the inner barrel 14, the flange 70 is deformed inwardly and forms a seal against the inner wall of the body 37, to seal the proximal end of the bore.

The outer surface of the plunger 16 may include one or more axially extending projections 90 (or ribs or grooves) to cooperate with the ribs or grooves 53 in the inner barrel 14 to align and locate the plunger 16 in the inner barrel 14. In conjunction with engagement of flange 70 against shoulder 50, the plunger 16 can be clipped into the inner barrel 14 as shown in FIG. 1.

A stop 82 is fitted into the open proximal end of the plunger 16 to close off the body 66, as seen in FIGS. 1-3. The stop 82 comprises a tubular part 92, configured to be a push fit with the interior of the body 66, and a flange 94 which provides a larger area for a user's thumb to press against when depressing the plunger 16 to operate the syringe 10.

A releasable stop means or clip 80 is releasably fitted around the proximal part of the body 66 of the plunger 16, as seen in FIGS. 1 and 2. As shown in FIG. 11, the releasable stop means 80 is preferably a resilient member which is generally C-shaped in cross section, dimensioned to securely fit around the plunger 16. Ridges 96 may protrude from the releasable stop means 80 to provide gripping features, enabling a user to push the releasable stop means 80 onto the plunger 16 and to pull it off again when required.

A cap 84 is releasably clipped over the nozzle 22 of the outer barrel 12 in order to seal the device 10 and prevent leakage of fluid through the nozzle 22, and to keep the nozzle 22 clean before use, as shown in FIG. 1.

When the device 10 is prepared and ready for use, a first composition is held in the outer barrel 12 in a first chamber defined by the smaller diameter portion 30. A second composition is held in the inner barrel 14 in a second chamber defined by the distal portion 48. The protection ring 18 is sealingly located on the inner barrel 14 to prevent leakage of the second composition which can exit the second chamber via the orifices 58. The plunger 16 is located in the proximal portion 46 of the inner barrel 14 with the flange 70 engaged against the shoulder 50. The inner barrel 14 and protection ring 18 are located in the proximal end of the outer barrel 12 with the protection ring 18 located in the larger diameter portion 26. The releasable stop means 80 is located around a proximal portion of the plunger 16. The nozzle 22 is covered by the cap 84. The distal end of the plunger 16 is closed by the stop 82. Thus, the device 10 is in the position shown in FIG. 1.

In order to deliver the first composition, a user first removes the cap 84 to expose the nozzle 22. The user then exerts pressure against the end flange 94 of the stop 82. Since the plunger 16 is located in the inner barrel 14 by engagement of the flange 70 against shoulder 50, the plunger 16 and inner barrel 14 move as one unit into the outer barrel 12. Thus, the first composition is displaced out of the first chamber and passes through the nozzle 22 to exit the device 10.

As the inner barrel 14 and plunger 16 move into the outer barrel 12, the protection ring 18 is retained at the proximal end of the outer barrel 12 in portion 26. Thus, the inner barrel 14 and plunger 16 move as one unit through the ring 18. The flexible annular flange 86 is pressed against the inner wall of the outer barrel 12, thereby preventing leakage of the composition in the second chamber into the first chamber. When the closed distal end 38 of the inner barrel 14 reaches the distal end of the outer barrel 12, the projection 40 is received in the nozzle 22 but does not completely block it. An annular chamber 98 is formed between the waisted portion 56 and the inner wall of the body 20 of the outer barrel 12. This communicates with the grooves 32 formed in the outer barrel 12. The flexible flange 86 is deformed by contact with the distal end wall of the outer barrel 12. This allows the second composition to exit the second chamber via orifices 58 and to flow via the annular channel 98 and the grooves 32 in order to reach the nozzle 22. At this point, the releasable stop means 80 prevents further movement of the plunger 16 into the inner barrel 14. Thus, when required the user removes the releasable stop means 80 and then exerts an increased pressure on the flange 68. This forces the base of the flange 70 to pass over the shoulder 50 and allows the plunger 16 to move into the distal portion 48 of the inner barrel 14. Thus, the second composition is displaced out of the second chamber in the inner barrel, via the orifices 58 and can flow through the annular channel 98 and grooves 32 to the nozzle 22 for delivery.

This releasable stop means for preventing movement of the plunger into the second chamber can include the removable clip as shown in FIGS. 1 and 11 and equivalents thereof. The releasable stop means for preventing movement of the plunger into the second chamber can also include one or more depressible flanges or one or more depressible rings on the body of the plunger; a twist lock mechanism that requires the plunger be rotated in relation to the barrel to allow further movement of the plunger into the barrel; one or more collapsible flanges or one or more collapsible rings that collapse at a significantly higher force than that required to move the plunger into the barrel in the absence of such collapsible flanges or rings; one or more flanges, hinges, or rings that are attached to the side of the body of the plunger and which must be pulled away from the body of the plunger to allow the plunger to move into the barrel, such one or more flanges, hinges, or rings may remain attached to at least a portion of the body of the plunger while still allowing the plunger to move into the barrel; one or more retractable flanges or rings that may be retracted by pulling on an element located inside the body of the plunger that is attached to said flanges or rings and protrudes from inside the body of the plunger.

At the very end of the stroke, as in FIG. 3, the nose 72 of the plunger 16 is located in the reduced diameter portion 54 at the distal end of the inner barrel 14 and the projection 74 is received in the recess 42.

In practise, the first composition delivered may be an antibiotic to treat mastitis in cattle and the second composition may be a teat seal. It is desirable to inject the antibiotic and then to massage the teat to ensure the medication accesses it properly, before injecting the seal. Therefore, the releasable stop means 80 provides the advantage that the seal cannot be dispensed unintentionally and will only be deliverable when a user is ready and has removed the releasable stop means 80.

Thus, a single device can be provided which contains first and second compositions in separate chambers and does not permit them to mix, but is able to sequentially deliver both compositions by one full stroke of the plunger.

1. A device comprising:
a first barrel defining a first chamber for holding a first composition, the first chamber having at least one side wall and a distal end wall defining a dispensing nozzle, wherein at least one flow channel is formed in the at least one side wall and distal end wall of the first chamber, extending across the distal end wall from the at least one side wall to the nozzle in helical fashion;
a second barrel defining a second chamber for holding a second composition, the second chamber having at least one side wall and a distal end wall, an annular seal at the distal end of the second barrel, and the at least one side wall of the second chamber defining at least one orifice proximal to the seal; and
a plunger operable to drive the second barrel into the first chamber to displace the first composition out of the first chamber through the nozzle, and to bring the annular seal of the second barrel into contact with the distal end wall of the first chamber, thereby to deform the seal and permit fluid communication between each orifice in the second barrel and each flow channel, wherein the plunger is subsequently moveable into the second chamber to displace the second composition out of the second chamber through each orifice and flow channel to the nozzle.

2. The device as claimed in claim 1, further comprising a plurality of flow channels arranged in three equi-spaced groups of flow channels.

3. The device as claimed in claim 2, further comprising releasable engagement means to engage the plunger with the second barrel such that when engaged the plunger and second barrel move as one unit to drive the second barrel into the first chamber, and when disengaged the plunger is able to move into the second chamber.

4. The device as claimed in claim 3, wherein the engagement means is releasable by exerting increased force on the plunger.

5. The device as claimed in claim 4, wherein the engagement means comprises cooperating structures on an inner surface of the second barrel and an outer surface of the plunger.

6. The device as claimed in claim 5, further comprising a closure member to prevent leakage of the second composition from the second barrel.

7. The device as claimed in claim 6, wherein the closure member comprises a tube sealingly located on the second barrel and the device further comprises retention structures to retain the tube and allow the second barrel to move through the tube and into the first chamber when driven by the plunger.

8. The device as claimed in claim 7, further comprising a releasable stop means for preventing movement of the plunger into the second chamber until the stop is released.

9. A device comprising:
a first barrel defining a first chamber for holding a first composition and a nozzle communicating with the first chamber;
a second barrel defining a second chamber for holding a second composition and at least one orifice communicating with the second chamber;
a plunger operable to drive the second barrel into the first chamber thereby to displace the first composition out of the first chamber through the nozzle and to bring each orifice into fluid communication with the nozzle, wherein the plunger is subsequently moveable into the second chamber to displace the second composition out of the second chamber through each orifice and the nozzle; and
further comprising a releasable stop means for preventing movement of the plunger into the second chamber until the stop is released.

10. The device as claimed in claim 9 wherein the releasable stop means comprises a resilient clip locatable around the plunger.

11. The device as claimed in claim 10, further comprising releasable engagement means to engage the plunger with the second barrel such that when engaged the plunger and second barrel move as one unit to drive the second barrel into the first chamber, and when the releasable stop means and engagement means are disengaged the plunger is able to move into the second chamber.

12. The device as claimed in claim 11, wherein the engagement means is releasable by exerting increased force on the plunger.

13. The device as claimed in claim 12, wherein the engagement means comprises cooperating structures on an inner surface of the second barrel and an outer surface of the plunger.

14. The device as claimed in claim 13, further comprising a closure member to prevent leakage of the second composition from the second barrel.

15. The device as claimed in claim 14, wherein the closure member comprises a tube sealingly located on the second barrel and the device further comprises retention structures to retain the tube and allow the second barrel to move through the tube and into the first chamber when driven by the plunger.

16. The device as claimed in claim 15, wherein the first chamber comprises a cylindrical wall and a distal end wall which defines the nozzle, and at least one flow channel is formed in the cylindrical wall and the distal end wall, extending from the cylindrical wall across the distal end wall to the nozzle in a helical fashion, wherein the second chamber also comprises a cylindrical wall and a distal end wall, and an annular seal is formed on the distal end of the second barrel, distal to the at least one orifice, wherein when the plunger drives the second barrel into the first chamber the annular seal is brought into contact with the distal end wall of the first chamber thereby deforming the seal and permitting fluid communication between each orifice and each flow channel.

17. A device comprising:
a first barrel defining a first chamber for holding a first composition and a nozzle communicating with the first chamber;
a second barrel defining a second chamber for holding a second composition and at least one orifice communicating with the second chamber;
a plunger operable to drive the second barrel into the first chamber thereby to displace the first composition out of the first chamber through the nozzle and to bring each orifice into fluid communication with the nozzle, wherein the plunger is subsequently moveable into the second chamber to displace the second composition out of the second chamber through each orifice and the nozzle; and
further comprising a tube sealingly locatable on the second barrel so as to cover each orifice, and retention structures operable to fix the tube relative to the first barrel and allow the second barrel to pass through the tube and into the first chamber when driven by the plunger.

18. The device as claimed in claim 17, further comprising releasable engagement means to engage the plunger with the second barrel such that when engaged the plunger and second barrel move as one unit to drive the second barrel into the first chamber, and when disengaged the plunger is able to move into the second chamber.

19. The device as claimed in claim 18, wherein the engagement means is releasable by exerting increased force on the plunger.

20. The device as claimed in claim 19, wherein the engagement means comprises cooperating structures on an inner surface of the second barrel and an outer surface of the plunger.

21. The device as claimed in claim 20, further comprising a releasable stop means for preventing movement of the plunger into the second chamber until it is released.

22. The device as claimed in claim 21, wherein the first chamber comprises a cylindrical wall and a distal end wall which defines the nozzle, and at least one flow channel is formed in the cylindrical wall and the distal end wall, extending from the cylindrical wall across the distal end wall to the nozzle in a helical fashion, wherein the second chamber also comprises a cylindrical wall and a distal end wall, an annular seal is formed on the distal end of the second barrel, distal to the at least one orifice, wherein when the plunger drives the second barrel into the first chamber the annular seal is brought into contact with the distal end wall of the first chamber thereby deforming the seal and permitting fluid communication between each orifice and each flow channel.
